(19) United States
(12) Patent Application Publication
(10) Pub. No.: US 2009/0143393 A1
(43) Pub. Date: Jun. 4, 2009

(54) METHOD OF STIMULATING HAIR GROWTH

(75) Inventors: Niall S. Doherty, Stonington, CT (US); Dennis A. Smith, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 12/368,329

(22) Filed: Feb. 10, 2009

Related U.S. Application Data
(63) Continuation of application No. 11/053,008, filed on Feb. 8, 2005, now abandoned.
(60) Provisional application No. 60/544,116, filed on Feb. 12, 2004.

(51) Int. Cl. A61K 31/501; A61Q 7/02

(52) U.S. Cl. 514/252.01

(57) ABSTRACT

The present invention is directed to the discovery that (3S,4R)-3,4-dihydro-4-(2,3-dihydro-2-methyl-3-oxopyridazin-6-yl)oxy-3-hydroxy-6-(3-hydroxyphenyl)sulphonyl-2,2,3-trimethyl-2H-benzo[b]pyran may be used to promote hair growth and alleviate alopecia.

This application claims priority of U.S. Provisional Application No. 60/544,116, filed Feb. 12, 2004.

### FIELD OF THE INVENTION

The invention is directed to the use of a compound to promote hair growth, alleviate alopecia, and to pharmaceutical formulations containing this compound.

### BACKGROUND OF THE INVENTION

Alopecia, or balding, is a common problem which medical science has yet to cure. While androgens are associated with balding, the physiological mechanism by which this hair loss occurs is not known. However, it is known that hair growth is altered in individuals afflicted with alopecia.

Hair does not grow continuously but undergoes cycles of activity involving periods of growth, rest, and shedding. The human scalp typically contains from 100,000 to 350,000 hair fibers or shafts, which undergo metamorphosis in three distinct stages:

(a) during the growth phase (anagen) the follicle (i.e. the hair root) penetrates deep into the dermis with the cells of the follicle dividing rapidly and differentiating in the process of synthesizing keratin, the predominant component of hair. In non-balding humans, this growth phase lasts from one to five years;
(b) the transitional phase (catagen) is marked by the cessation of mitosis and lasts from two to three weeks; and
(c) the resting phase (telogen) in which the hair is retained within the scalp for up to 12 weeks, until it is displaced by new follicular growth from the scalp below.

In humans, this growth cycle is not synchronized. An individual will have thousands of follicles in each of these three phases. However, most of the hair follicles will be in the anagen phase. In healthy young adults, the anagen to telogen ratio can be as high as 9 to 1. In individuals with alopecia, this ratio is reduced to as low as 2:1.

Androgenetic alopecia arises from activation of an inherited sensitivity to circulating androgenic hormones. It is the most common type of alopecia. It affects both men (50%) and women (30%), primarily of Caucasian origin. Gradual changes in the width and length of the hair shaft are experienced over time and with increasing age, prematurely in some. Terminal hair is gradually converted to short, wispy, colorless vellus hair. As a consequence, men in their 20's and women in their 30's and 40's begin to notice their hair becoming finer and shorter. In males, most of the hair loss occurs at the crown of the head. Females experience a thinning over their entire scalp. As discussed above, the anagen to telogen ratio is reduced significantly, resulting in less hair growth.

Minoxidil, a potassium channel opener, promotes hair growth. Minoxidil is available commercially in the United States under the trademark, Rogaine®. While the exact mechanism of action of minoxidil is unknown, its impact on the hair growth cycle is well documented. Minoxidil promotes the growth of the hair follicle and increases the period of time that the hair follicle is in the anagen phase (i.e. increases the anagen to telogen ratio).

While minoxidil promotes hair growth, the cosmetic efficacy of this growth can vary widely. For example, Roenigk reported the results of a clinical trial involving 83 males who used a topical solution of 3% minoxidil for a period of 19 months. Hair growth occurred in 55% of the subjects. However, only 20% of the subjects considered the growth to be cosmetically relevant. (Clin. Res., 33, No. 4, 914A, 1985). Tosti reported cosmetically acceptable regrowth in 18.1% of his subjects. (Dermatologica, 173, No. 3, 136-138, 1986). Thus, the need exists in the art for compounds having the ability produce higher rates of cosmetically acceptable hair growth in patients with alopecia.

### SUMMARY OF THE INVENTION

In accordance with the present invention, a new method for promoting hair growth has been discovered. The method comprises the administration of a compound of the formula:

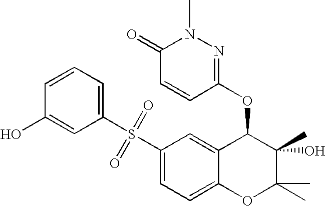

a salt thereof, a solvate thereof, or an admixture thereof, to a mammal exhibiting alopecia. Typically, the mammal will be a human suffering from alopecia, especially androgenetic alopecia. However the compound may be administered to any mammal that would benefit by having the growth of their hair stimulated.

A further embodiment of the invention is directed to a topical formulation containing an effective amount of the compound in admixture with a dermatologically acceptable carrier. This formulation will be applied to the scalp of a human, for a sufficient period of time to promote hair growth.

An additional embodiment of the invention is directed to a pharmaceutical formulation containing the compound, packaged for retail distribution, associated with instructions advising the consumer how to use the product in order to stimulate the growth of hair.

### DETAILED DESCRIPTION OF THE INVENTION

### A) Definitions

As used throughout this application, including the claims, the following terms have the meanings defined below, unless specifically indicated otherwise. The plural and singular should be treated as interchangeable, other than the indication of number
a. “Mammal” includes humans, primates such as stump-tailed macaques, companion animals such as dogs, cats, gerbils, etc. and livestock such as cattle, swine, horses, llamas, and sheep.
b. “Promoting hair growth” includes stimulating an increase in total hair mass and/or length. Such increase includes increased length and/or growth rate of hair shafts (i.e. follicles), increased number of hairs, and/or increased hair thickness. Some or all of the above end results can be achieved by prolonging or activating anagen, the growth phase of the hair cycle, or by shortening or delaying the catagen and telogen phases. “Promoting hair growth” should also be considered to include preventing, arresting, decreasing, delaying and/or reversing hair loss.
c. “Alopecia,” as used herein, encompasses partial or full baldness, hair loss, and/or hair thinning.
d. “Treating or alleviating alopecia” refers to promoting hair growth in mammals who have experienced, or are considered at risk for experiencing, alopecia.
e. “Pharmaceutically acceptable” means suitable for use in mammals.
f. “any reference to the compound of Formula I shall at all times be understood to include all active forms of the compound, including, for example, the free form thereof, e.g., the free acid or base form, and also, all prodrugs, polymorphs, hydrates, solvates, tautomers, stereoisomers, e.g., diastereomers and enantiomers, and the like, and all pharmaceutically acceptable salts, and admixtures of such physical forms, unless specifically stated otherwise. All of these forms are described in U.S. Pat. No. 5,912,244, the contents of which are hereby incorporated by reference. It will also be appreciated that suitable active metabolites of such compound, in any suitable form, are also included herein.
g. “solvate” is a crystalline form of a compound or salt thereof, containing one or more molecules of a solvent of crystallization, i.e., a compound of Formula I or a salt thereof, containing solvent combined in the molecular form. A “hydrate” is a solvate in which the solvent is water.
h. “polymorph” is a compound or salt thereof, such as the compound of Formula I or a salt thereof, which occurs in at least one crystalline form.
i. “pharmaceutically acceptable salts” is intended to refer to either “pharmaceutically acceptable acid addition salts” or “pharmaceutically acceptable basic addition salts”. “Salts” is intended to refer to “pharmaceutically acceptable salts” or to salts suitable for use in industrial processes, that might not be pharmaceutically acceptable.
j. “pharmaceutically acceptable acid addition salts” is intended to apply to any non-toxic organic or inorganic acid addition salt of the base compound represented by Formula I or any of its intermediates. Illustrative inorganic acids which form suitable salts include hydrochloric, hydrobromic, sulphuric, and phosphoric acid and acid metal salts such as sodium monohydrogen orthophosphate, and potassium hydrogen sulfate. Illustrative organic acids, which form suitable salts include the mono-, di-, and tricarboxylic acids. Illustrative of such acids are for example, acetic, glycolic, lactic, pyruvic, malonic, succinic, glutaric, fumaric, malic, tartaric, citric, ascorbic, maleic, hydroxymaleic, benzoic, hydroxy-benzoic, phenylacetic, cinnamic, salicylic, 2-phenoxybenzoic, p-toluenesulfonic acid, and sulfonic acids such as methane sulfonic acid and 2-hydroxyethane sulfonic acid. Such salts can exist in either a hydrated or substantially anhydrous form. In general, the acid addition salts of these compounds are soluble in water and various hydrophilic organic solvents.
k. “pharmaceutically acceptable basic addition salts” is intended to apply to any non-toxic organic or inorganic basic addition salts of the compound represented by Formula I, or any of its intermediates. Illustrative bases which form suitable salts include alkali metal or alkaline-earth metal hydroxides such as sodium, potassium, calcium, magnesium, or barium hydroxides; ammonia, and aliphatic, alicyclic, or aromatic organic amines such as methylamine, dimethylamine, trimethylamine, and picoline.
l. “prodrug” refers to compounds that are rapidly transformed in vivo to yield the parent compound of the above formula, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference.
m. “compound of Formula I”, “compounds of the invention”, and “compounds” are used interchangeably throughout the application and should be treated as synonyms.

### B) The Compound

The compound useful in the present invention is (3S,4R)-3,4-dihydro-4-(2,3-dihydro-2-methyl-3-oxopyridazin-6-yl)oxy-3-hydroxy-6-(3-hydroxyphenyl)sulphonyl-2,2,3-trimethyl-2H-benzo[b]pyran (hereinafter the “compound”). It may be represented by the formula immediately below:

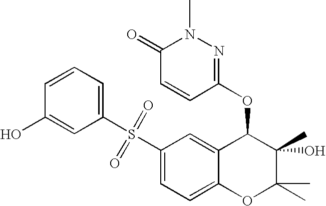

This compound and methods for its preparation are described in U.S. Pat. No. 5,912,244, the contents of which are hereby incorporated by reference. Example 7 of the '244 patent exemplifies one method for producing this compound.

In addition to the compound of Formula I above, the '244 patent discloses a genus of benzopyran derivatives. The '244 patent discloses that these compounds are potassium channel openers that exhibit smooth muscle relaxant activity. The '244 patent also discloses that these compounds may be used to treat diseases associated with altered tone or motility of smooth muscles. Examples of such conditions include chronic obstructive airway disease, asthma, urinary incontinence, hypertension, myocardial ischemia, cerebral ischemia, glaucoma, and male pattern baldness.

An assay for assessing the compounds potency as potassium channel openers is described in column 9 of the '244 application, at lines 3-41. Data for selected compounds is depicted in the Table bridging columns 30 and 31. No data is presented for the product of Example 7, which is the compound of Formula I.

### C) Pharmacology and Medical Uses

As noted above, the compound of Formula I is a potassium channel opener. It has been discovered that this compound has unexpected activity in the promotion of hair growth, when compared with other potassium channel openers. The compound will stimulate the growth of the hair follicle, increase the number of follicles in the anagen phase and increase the period of time that follicles remain in the anagen phase (i.e. increase the anagen to telogen ratio).

The compound may be used to promote hair growth in humans. Thus it may be used to alleviate alopecia. In order to alleviate the subject's alopecia, the compound needs to be administered in a quantity sufficient to promote hair growth. This amount can vary depending upon the type of alopecia being treated, the severity of the patient's alopecia, the patient, the duration of the alopecia, the route of administration, and the presence of other underlying disease states within the patient, etc. When administered systemically, the compound typically exhibits its effect at a dosage range of from about 0.1 mg/kg/day to about 100 mg/kg/day. Repetitive daily administration may be desirable and will vary according to the conditions outlined above

The compound may be administered by a variety of routes. It may be administered orally. It may also be administered parenterally (i.e. subcutaneously, intravenously, intramuscularly, intraperitoneally, or intrathecally), rectally, or topically.

In a typical embodiment, the compound is administered topically to promote hair growth. The compound will generally be applied directly to the scalp, especially to those areas in which hair is absent, or thinning. The dose will vary, but as a general guideline, the compound will be present in a dermatologically acceptable carrier in an amount of from 0.01 to 10 w/w %, and the dermatological preparation will be applied to the affected area from 1 to 4 times daily. More typically, the compound will be present in a quantity of from 1 to 3 w/w %, and the compound will be applied once or twice daily. “Dermatologically acceptable” refers to a carrier which may be applied to the skin or hair, and which will allow the drug to diffuse to the site of action.

In a further embodiment, the compound can also be used in patients who have not yet experienced hair loss, but believe that they are at risk of experiencing alopecia. Examples of such patients include those who will be undergoing cancer chemotherapy with a drug regimen known to induce alopecia. Young adults experiencing mental distress at the thought of balding, especially those with a family history of baldness, may also benefit from such prophylactic treatment. Such prophylactic treatment is encompassed by the term “promoting hair growth”.

The most common type of alopecia is androgenetic alopecia. This condition is also commonly referred to as male pattern baldness and female pattern baldness. The compound may be used to promote hair growth in individuals suffering from this type of alopecia.

Anagen effluvium, is hair loss due to chemicals or radiation, such as chemotherapy or radiation treatment for cancer. It is also commonly referred to as “drug induced” or “radiation induced” alopecia. The compound may be used in this condition.

Alopecia areata is an autoimmune disorder which initially presents with hair loss in a rounded patch on the scalp. It can progress to the loss of all scalp hair, which is known as alopecia totalis and to the loss of all scalp and body hair, which is known as alopecia universalis. The compound may be utilized for these types of alopecia.

Traumatic alopecia is the result of injury to the hair follicle. It is also commonly referred to as “scarring alopecia”. Psychogenic alopecia occurs due to acute emotional stress. By inducing anagen, the compound can be beneficial in these types of alopecia as well. Thus, the invention should not be construed as being limited to treating androgenetic alopecia. The compound can be used to alleviate any type of hair loss.

The compound may be used to promote hair growth in other mammals besides humans. For example, the compound may be used with farm animals such as sheep, in which fur (hair) growth would exhibit an economic benefit. The compound may also be used to stimulate hair growth in companion animals such as dogs, cats, gerbils, etc. The dosages required to obtain this effect will fit within the guidelines described above. Likewise, the compound may be administered using formulations typically used for veterinary applications, taking into account the type of animal being treated. Other applications of the compound to promote hair growth will become readily apparent to one skilled in the art based upon the disclosure of this application and should be considered to be encompassed by the claims.

### D) Formulations

If desired, the compound can be administered directly without any carrier. However, to ease administration, it will typically be formulated with at least one pharmaceutically acceptable or cosmetically acceptable carrier (herein collectively described as a “carrier”). The term “carrier,” as used herein, means one or more compatible solid or liquid fillers, diluents, vehicles or encapsulating substances, which are suitable for administration to a mammal. The term “compatible,” as used herein, means that the components of the composition are capable of being comingled with a compound as described herein, and with each other, in a manner such that there is no interaction that would substantially reduce the efficacy of the composition under ordinary use situations. Carriers must, of course, be of sufficiently high purity and sufficiently low toxicity to render them suitable for administration to the mammal (preferably the human being) being treated. The carrier itself can be inert or it can possess pharmaceutical and/or cosmetic benefits of its own.

The compound may be formulated in any of a variety of suitable forms, for example, oral, topical or parenteral administration. Standard pharmaceutical formulation techniques may be used, such as those disclosed in Remington's Pharmaceutical Sciences, Mack Publishing Company, Easton, Pa. (1990).

Depending upon the particular route of administration, a variety of carriers well known in the art may be used. These include solid or liquid fillers, diluents, hydrotropes, surface-active agents and encapsulating substances. Optional pharmaceutically active or cosmetically active materials may be included which do not substantially interfere with the activity of the compound used in the methods of the present invention. The amount of carrier employed in conjunction with the compound used in the methods of the present invention is sufficient to provide a practical quantity of material for administration per unit dose of the compound. Techniques and compositions for making dosage forms useful in the methods of the present invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

Typically, the compound is administered topically. The carrier of the topical composition may aid penetration of the compound into the skin to reach the environment of the hair follicle. Such topical compositions may be in any form including, for example, solutions, oils, creams, ointments, gels, lotions, pastes, shampoos, leave-on and rinse-out hair conditioners, milks, cleansers, moisturizers, sprays, aerosols, skin patches and the like.

A variety of carrier materials well known in the art for topical application, such as, for example, water, alcohols, aloe vera gel, allantoin, glycerine, vitamin A and E oils, mineral oil, propylene glycol, and the like can be used to prepare such formulations. The references discussed above disclose a number of excipients that can be used to prepare such topical dosage forms.

The compound may also be administered topically in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. A potential formulation for topical delivery of the compound used in the methods of the present invention utilizes liposomes such as described in Dowton et al., “Influence of Liposomal Composition on Topical Delivery of Encapsulated Cyclosporin A: I. An in vitro Study Using Hairless Mouse Skin”, S.T.P. Pharma Sciences, Vol. 3, pp. 404-407 (1993); Wallach and Philippot, “New Type of Lipid Vesicle: Novasome®”, Liposome Technology, Vol. 1, pp. 141-156 (1993); U.S. Pat. No. 4,911,928; and U.S. Pat. No. 5,834,014.

Carriers for systemic administration include, for example, sugars, starches, cellulose and its derivatives, malt, gelatin, talc, calcium sulfate, vegetable oils, synthetic oils, polyols, alginic acid, phosphate buffer solutions, emulsifiers, isotonic saline and pyrogen-free water. Suitable carriers for parenteral administration include, for example, propylene glycol, ethyl oleate, pyrrolidone, ethanol and sesame oil.

Various oral dosage forms can be used, including such solid forms as tablets, capsules, granules and bulk powders. These oral forms comprise an effective amount, usually at least about 5% of the compound. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

Orally administered compositions also include liquid solutions, emulsions, suspensions, powders, granules, elixirs, tinctures, syrups and the like. The carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, Avicel RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents or colorants as described above.

Other compositions useful for attaining systemic delivery of the compound useful in the methods of the present invention include sublingual, buccal and nasal dosage forms. Such compositions typically comprise one or more soluble filler substances such as sucrose, sorbitol and mannitol; and binders such as acacia, microcrystalline cellulose, carboxymethyl cellulose and hydroxypropyl methylcellulose. Glidants, lubricants, sweeteners, colorants, antioxidants and flavoring agents described above may also be included.

The dosage forms described above may be packaged for retail distribution directly to the consumer (i.e. an article of manufacture or kit). Such articles will be labeled and packaged in a manner advising the patient how to use the product to promote hair growth. Such instructions will include the duration of treatment, dosing schedule, precautions, etc. These instructions may be in the form of pictures, written instructions, or a combination thereof. They may be printed on the side of the packaging, be an insert, or any other form of communication appropriate for the retail market.

The compound of Formula I may also be admixed with any inert carrier and utilized in laboratory assays in order to determine the concentration of the compounds within the serum, urine, etc., of the patient as is known in the art. The compound may also be used as a research tool.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention. The following examples and biological data are being presented in order to further illustrate the invention. This disclosure should not be construed as limiting the invention in any manner.

### E) Examples

### A) Telogen Conversion Assay

The Telogen Conversion Assay measures the potential of a compound (hereinafter referred to as the “test compound”) to convert mice in the resting stage of the hair growth cycle (“telogen”) to the active stage of the hair growth cycle (“anagen”). This assay takes advantage of the fact that the fur (i.e. hair) of 40-day-old C3H/HeN mice is in the telogen phase. This phase usually continues until about 75 days of age, at which point, anagen naturally occurs in these animals. In this assay, selected areas of 40-day-old mice (approximate) are shaved, contacted with a test agent, or a control, and the difference in the rate of hair growth is measured (i.e. induction of the anagen phase). The first sign of anagen is the darkening of skin color as melanocytes in the follicles start to synthesize melanin, in preparation for the production of pigmented hairs.

Test Compounds

As part of a research project, selected potassium channel openers were evaluated in the telogen conversion assay. All of the compounds had previously been described in the literature as potassium channel openers and were based upon a common benzopyran nucleus (See U.S. Pat. Nos. 5,912,244 and 5,677,324). The test compounds are described below in Table A.

TABLE A

| | | Literature |
|---|---|---|
| Compound | Structure | citation |
| #1 Invention | 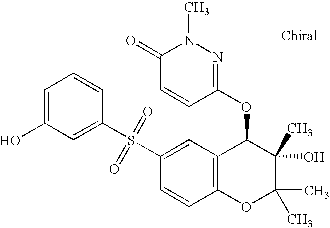 | Example 7 of U.S. Pat. No. 5,912,244 |
| #2 | 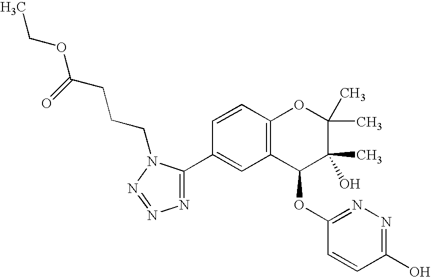 | Example 1 of U.S. Pat. No. 5,677,324 |
| #3 | 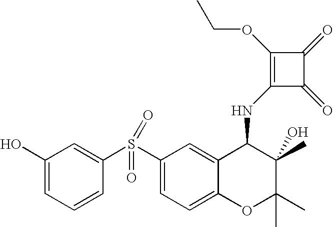 | Example 2 of U.S. Pat. No. 5,912,244 |
| #4 | 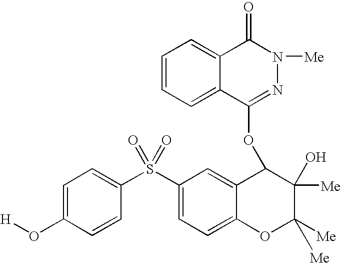 | Example 10 of U.S. Pat. No. 5,912,244 |
| #5 | 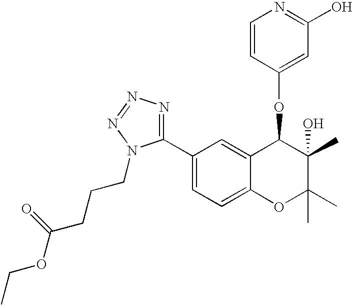 | Example 2 Example 1 of U.S. Pat. No. 5,677,324 |
| #6 | 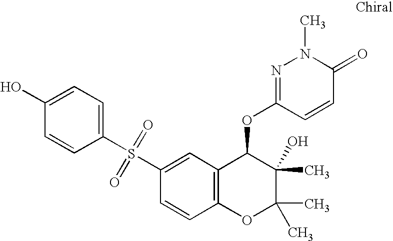 | Example 5 of U.S. Pat. No. 5,912,244 |
| #7 | 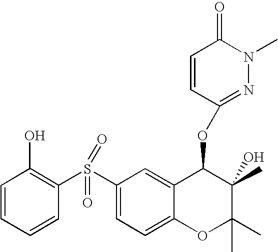 | Example 8 of U.S. Pat. No. 5,912,244 |

The compounds were chosen for testing in the telogen conversion assay on the basis of their in-vitro activity as potassium channel openers. Each compound had sufficient activity to lead one skilled in the art to expect that the compounds would have a significant potential for exhibiting activity in relevant animal models.

### Experimental Procedures:

Female C3H/HeN mice, 7 weeks old (Charles River Laboratories, Raleigh, N.C.) were used for the study. Fur was clipped from the dorsal region of the mice prior to initiation of the study. Only mice with pink skin, a visual indication of the telogen phase, were selected for inclusion in the study.

Test compounds (from Table A) were dissolved in a vehicle consisting of propylene glycol (30%) and ethanol (70%). A test compound dissolved in vehicle, or a vehicle control (30/70 propylene glycol/ethanol, unless otherwise specified) was applied topically to the clipped dorsal region of the mice in each test group (7-10 mice) in a volume of 20 μl/cm². Concentration of drug varied as shown in Tables 1-15 below. Treatments were applied once daily for 5 days.

The treatment area was observed and graded for signs of hair growth. The hair growth response was quantified by recording, for each animal, the day on which signs of hair growth first appeared over the treated area. The first sign of anagen was the darkening of skin color as melanocytes in the follicles started to synthesize melanin. The response time was measured as the number of days between initiation of treatment and when hair growth was present in 50% of the mice in a given group. The mice were observed for up to 35 days, or longer.

### Results

The results are reported as the number of days following initiation of treatment when hair growth appeared in 50% of the mice in a given group. Tables 1-15 report the results of these experiments.

Considerable variation was encountered in the results obtained in these experiments, based upon the day when anagen was observed in 50% of the animals in the vehicle control group. For example in Experiment 2, anagen was observed in 50% of the animals in the vehicle control group on day 25. In Experiment 11, it took 56 days for the control group to reach the 50^{th} percentile.

Based upon this variability, the inventors have concluded that a number of experiments were terminated early, i.e. prior to the day on which hair growth was present in 50% of the test animals. Those experiments that were terminated early should not be evaluated in the same manner as those in which the control group was allowed to reach the 50^{th} percentile.

In those experiments that were terminated early, one cannot conclude that a compound is inactive merely because it did not induce anagen (i.e. hair growth) prior to the termination of the experiment. It is possible that if the experiment had been allowed to proceed to completion, i.e. the day on which the control group reached the 50^{th} percentile, the compound may have induced anagen earlier than the control group.

In those experiments in which anagen was observed with a test compound despite the early termination, one can conclude that the compound is active. One can potentially also detect differences in the efficacy of two different compounds based upon when the compound induced anagen in 50% of the animals. Thus, Experiments 1, 3, 5, 6, 8, 9, 10, and 15, which were terminated early, should be evaluated in light of these comments.

### Experiment 1

In this experiment, Compound #1 was tested in the telogen conversion assay. The following results were observed:

TABLE 1
Compound #1

| Concentration | Results |
|---|---|
| 1.0 w/v % | >35 |
| Vehicle | >35 |

This experiment was terminated to soon to draw any conclusions regarding the results.

### Experiment 2

In this experiment, Compound #'s 1 and 6 were evaluated in the telogen conversion assay. The following results were obtained:

TABLE 2

| | |
|---|---|
| Concentration | Results |

Compound #1

| | |
|---|---|
| 0.3 w/v % | 11 |
| 1.0 w/v % | 11 |
| Vehicle | 25 |

Compound #6

| | |
|---|---|
| 0.3 w/v % | 18 |
| 1.0 w/v % | 30 |

Mice treated with Compound #1 exhibited signs of anagen sooner than those receiving Compound #6.

### Experiment 3

In this protocol, Compound #'s 1 and 2 were evaluated in the telogen conversion assay. The following results were obtained:

TABLE 3

| | |
|---|---|
| Concentration | Results |

Compound #1

| | |
|---|---|
| 0.1 w/v % | >35 |
| 0.3 w/v % | >35 |
| 1.0 w/v % | >35 |
| Vehicle | >35 |

Compound #2

| | |
|---|---|
| 0.1 w/v % | >35 |
| 0.3 w/v % | >35 |
| 1.0 w/v % | >35 |
| 2.5 w/v % | >35 |

This experiment was terminated early, precluding one from drawing any conclusions regarding the relative efficacy of these compounds.

### Experiment 4

In this protocol, compound #1 was tested in the telogen conversion assay. The following results were observed:

TABLE 4
Compound #1

| Concentration | Results |
|---|---|
| 0.3 w/v % | 26 |
| 1.0 w/v % | 26 |
| Vehicle | >33 |

Despite the early termination of this experiment, Compound #1 induced anagen at each of the test concentrations.

### Experiment 5

In this protocol, Compound #'s 1 and 6 were evaluated in the telogen conversion assay. The following results were obtained:

TABLE 5

| | |
|---|---|
| Concentration | Results |

Compound #1

| | |
|---|---|
| 0.03 w/v % | >35 |
| 0.1 w/v % | >35 |
| 0.3 w/v % | >35 |
| Vehicle | >35 |

Compound #6

| | |
|---|---|
| 0.03 w/v % | >35 |
| 0.1 w/v % | >35 |
| 0.3 w/v % | 23 |

Despite the early termination of this experiment, compound #6 induced anagen at the highest concentration tested (0.3%).

### Experiment 6

In this protocol, Compound #'s 1 and 6 were evaluated in the telogen conversion assay. The following results were obtained:

TABLE 6

| | |
|---|---|
| Concentration | Results |

Compound #1

| | |
|---|---|
| 0.003 w/v % | >35 |
| 0.03 w/v % | >35 |
| 0.3 w/v % | 28 |
| Vehicle | >35 |

Compound #6

| | |
|---|---|
| 0.003 w/v % | >35 |
| 0.03 w/v % | >35 |
| 0.3 w/v % | >35 |

Compound #1 induced anagen despite the early termination of the experiment, whereas no effect was seen from Compound #6 up to day 35.

### Experiment 7

In this protocol, Compound #'s 1 and 6 were evaluated in the telogen conversion assay. The following results were obtained:

TABLE 7

| | |
|---|---|
| Concentration | Results |

Compound #1

| | |
|---|---|
| 0.003 w/v % | 26 |
| 0.03 w/v % | 24 |
| 0.3 w/v % | 19 |
| Vehicle | 29 |

Compound #6

| | |
|---|---|
| 0.003 w/v % | 31 |
| 0.03 w/v % | >33 |
| 0.3 w/v % | >33 |

This experiment was allowed to proceed to completion. Compound #1 induced anagen at all of the concentrations tested. Compound #6 did not induce anagen prior to the control group and thus could be concluded to be inactive in this experiment.

### Experiment 8

In this protocol, Compound #'s 1, 3, 4, 5, and 7 were evaluated in the telogen conversion assay. The following results were obtained:

TABLE 8

| | |
|---|---|
| Concentration | Results |

Compound #1

| | |
|---|---|
| 0.3 w/v %¹ | 21 |
| 1.0 w/v %¹ | 21 |
| 1.0 w/v % | 18 |
| Vehicle¹ | 28 |
| Vehicle | >35 |

Compound #3

| | |
|---|---|
| 0.3 w/v %¹ | >35 |
| 1.0 w/v %¹ | >35 |

Compound #4

| | |
|---|---|
| 0.3 w/v %¹ | >35 |
| 1.0 w/v %¹ | >35 |

Compound #5

| | |
|---|---|
| 0.3 w/v %¹ | >35 |
| 1.0 w/v %¹ | >35 |

Compound #7

| | |
|---|---|
| 0.3 w/v %¹ | 21 |
| 1.0 w/v %¹ | 21 |

¹Solvent system contains polyethylene glycol 30 v/v %, ethanol 30 v/v %, and transcutanol 40 v/v %.

In this experiment two different vehicles were used. One experiment used a solvent containing transcutanol (a penetration enhancer), ethanol and polyethylene glycol. The other solvent was a 30:70 admixture of propylene glycol and ethanol. All compounds were prepared in the transcutol, ethanol, polypropylene glycol vehicle and should be compared with the control group that was treated with this vehicle.

The experiment was continued for a sufficient period of time to allow the transcutol, ethanol, polypropylene glycol control group to reach the 50% mark. Compound #1 exhibited activity at all doses tested in this vehicle. Compound #'s 3, 4, and 5 were inactive in these experiments. Compound #7 did exhibit activity in this experiment.

### Experiment 9

In this experiment, Compound #'s 1 and #7 were evaluated in the telogen conversion assay. The following results were obtained:

TABLE 9

| | |
|---|---|
| Concentration | Results |

Compound #1

| | |
|---|---|
| 0.03 w/v % | >33 |
| 0.3 w/v % | >33 |
| 1.0 w/v % | >33 |
| Vehicle | >33 |

Compound #7

| | |
|---|---|
| 0.03 w/v % | >33 |
| 0.3 w/v % | >33 |
| 1.0 w/v % | >33 |

This experiment was concluded too early to allow one to draw any conclusions from the results.

### Experiment 10

In this experiment, Compound #'s 1 and 7 were evaluated in the telogen conversion assay. The following results were obtained

TABLE 10

| | |
|---|---|
| Concentration | Results |

Compound #1

| | |
|---|---|
| 0.1 w/v % | >34 |
| 0.3 w/v % | >34 |
| Vehicle | >34 |

Compound #7

| | |
|---|---|
| 0.1 w/v %¹ | >34 |
| 0.3 w/v %¹ | >34 |
| 0.1 w/v % | >34 |
| 0.3 w/v % | >34 |

¹Solvent system contains polyethylene glycol 30 v/v %, ethanol 30 v/v %, and transcutanol 40 v/v %.

This experiment was also concluded too early to allow one to draw any conclusions from the results.

### Experiment 11

In this experiment, Compound #1 was evaluated in the telogen conversion assay. The following results were obtained:

TABLE 11
Compound #1

| Concentration | Results |
|---|---|
| 0.3 w/v % | 25 |
| Vehicle | 56 |

This experiment was carried for a sufficient period of time to allow the control group to reach the 50^{th} percentile. Compound #1 induced anagen.

### Experiment 12

In this experiment, Compound #1 was evaluated in the telogen conversion assay. The following results were obtained:

TABLE 12
Compound #1

| Concentration | Results |
|---|---|
| 1.0 w/v % | 39 |
| Vehicle | 37 |

This experiment was carried for a sufficient period of time to allow the control group to reach the 50^{th} percentile. Compound #1 did not induce anagen in this experiment.

### Experiment 13

In this experiment, Compound # 1 was evaluated in the telogen conversion assay. The following results were obtained:

TABLE 13
Compound #1

| Concentration | Results |
|---|---|
| 1.0 w/v % | 14 |
| Vehicle | 28 |

This experiment was carried for a sufficient period of time to allow the control group to reach the 50^{th} percentile. Compound #1 induced anagen.

### Experiment 14

In this experiment, Compound #1 was evaluated in the telogen conversion assay. The following results were obtained:

TABLE 14
Compound #1

| Concentration | Results |
|---|---|
| 1.0 w/v % | 25 |
| Vehicle | 29 |

This experiment was carried for a sufficient period of time to allow the control group to reach the 50^{th} percentile. Compound #1 induced anagen.

### Experiment 15

In this protocol, Compound #'s 1, 6, and 7 were evaluated in the telogen conversion assay. The following results were obtained:

TABLE 15

| | |
|---|---|
| Concentration | Results |

Compound #1

| | |
|---|---|
| 0.3 w/v % | 16 |
| 1.0 w/v % | 23 |
| Vehicle | >30 |

Compound #6

| | |
|---|---|
| 0.03 w/v % | >30 |
| 0.3 w/v % | 14 |
| 1.0 w/v % | >30 |

Compound #7

| | |
|---|---|
| 0.03 w/v % | >30 |
| 0.3 w/v % | >30 |
| 1.0 w/v % | >30 |

This experiment was terminated early. Compound #1 induced anagen at both of the concentrations tested, despite of the early termination. Compound #7 showed no effect at the time of termination. Compound #6 induced anagen when applied at a concentration of 0.3 w/v %.

### SUMMARY

(3S,4R)-3,4-dihydro-4-(2,3-dihydro-2-methyl-3-oxopyridazin-6-yl)oxy-3-hydroxy-6-(3-hydroxyphenyl)sulphonyl-2,2,3-trimethyl-2H-benzo[b]pyran (i.e. Compound #1) was tested in the telogen conversion assay on 15 different occasions, at a dose of once daily for 5 days. Eight of the experiments were terminated early, complicating evaluation of the results. Compound #1 showed the highest relative efficacy in the model, when compared with the other potassium channel openers listed in Table A. This outcome was unexpected. Based upon its in-vitro activity as a potassium channel opener, there was no basis for predicting that this compound would exhibit superior activity in the telogen conversion assay.

What is claimed is:

1. Use of a compound of the formula: 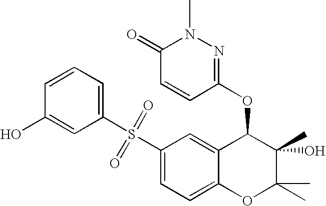
a pharmaceutically acceptable salt thereof, or a solvate thereof, in the manufacture of a medicament for alopecia.

2. The use of claim 1 in which said alopecia is selected from the group consisting of alopecia areata, anagen effluvium, self-induced hair loss, telogen effluvium, scarring alopecia and androgenetic alopecia.

3. The use of claim 1 in which said alopecia is androgenetic alopecia.

4. The use of any of claims 1-3 in which said medicament is topical.

5. Use of a compound of the formula: 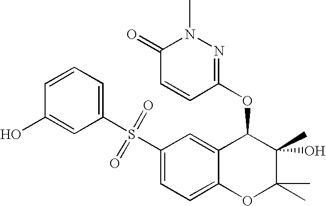
a pharmaceutically acceptable salt thereof, or a solvate thereof, in the manufacture of a medicament for promoting hair growth.

6. The use according to claim 5 in which said medicament is topical.

7. A topical pharmaceutical formulation comprising a compound of the formula: 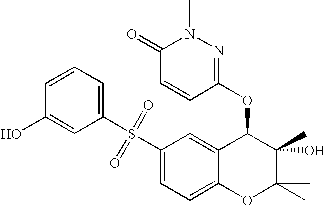
a pharmaceutically acceptable salt thereof, or a solvate thereof, in admixture with at least one pharmaceutically acceptable topical carrier.

8. An article of manufacture comprising a pharmaceutical formulation according to claim 7 packaged for retail distribution, in association with instructions explaining how to use said formulation to treat alopecia.

9. An article of manufacture comprising a pharmaceutical formulation according to claim 7 packaged for retail distribution, in association with instructions explaining how to use said formulation to promote hair growth.

10. Use of a compound of the formula: 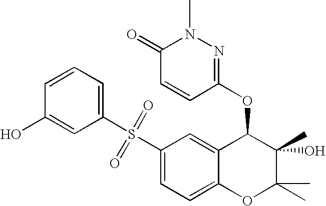
a pharmaceutically acceptable salt thereof, or a solvate thereof, in the manufacture of a topical medicament for androgenetic alopecia.

11. Use of a compound of the formula: 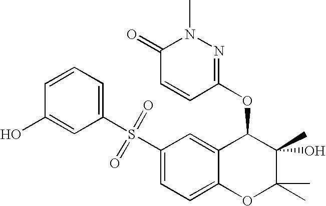
a pharmaceutically acceptable salt thereof, or a solvate thereof, in the manufacture of a topical medicament for inducing anagen.
